(19) United States
(12) Patent Application Publication
(10) Pub. No.: US 2013/0316918 A1
(43) Pub. Date: Nov. 28, 2013

(54) MODEL-BASED RESIDUAL CORRECTION OF INTENSITIES

(71) Applicants: Ming Jiang, Foster City, CA (US); Chengyong Yang, Foster City, CA (US); Eugene Wang, Arcadia, CA (US)

(72) Inventors: Ming Jiang, Foster City, CA (US); Chengyong Yang, Foster City, CA (US); Eugene Wang, Arcadia, CA (US)

(73) Assignee: LIFE TECHNOLOGIES CORPORATION, Carlsbad, CA (US)

(21) Appl. No.: 13/989,026

(22) PCT Filed: Nov. 22, 2011
(86) PCT No.: PCT/US2011/061889

§ 371 Date: Aug. 12, 2013

Related U.S. Application Data
(60) Provisional application No. 61/416,256, filed on Nov. 22, 2010.
(60) Provisional application No. 61/478,229, filed on Apr. 22, 2011.

(51) Int. Cl. C12Q 1/68

(52) U.S. Cl. CPC C12Q 1/6874
USPC 506/2; 506/38

(57) ABSTRACT

A method for improving color calls or base calls utilizes current and prior cycle multi-channel intensity data from a sequencing run to model residual cycle buildup. The model is applied to correct the multi-cycle channel intensity for the current cycle. The corrected multi-cycle channel intensity is used for color calls or base calls for the current cycle.

### CROSS-REFERENCE TO RELATED APPLICATIONS

This application is a U.S. National Application filed under 35 U.S.C. 371 of International Application No. PCT/US2011/061889, filed Nov. 22, 2011 which claims priority to U.S. Ser. No. 61/416,256, filed Nov. 22, 2010, and U.S. Ser. No. 61/478,229, filed Apr. 22, 2011, the disclosures of which are hereby incorporated herein by reference in their entirety as if set forth fully herein.

### FIELD

The present disclosure is directed toward polynucleotide sequencing.

### INTRODUCTION

Nucleic acid sequencing techniques are of major importance in a wide variety of fields ranging from basic research to clinical diagnosis. The results available from such technologies can include information of varying degrees of specificity. For example, useful information can consist of determining whether a particular polynucleotide differs in sequence from a reference polynucleotide, confirming the presence of a particular polynucleotide sequence in a sample, determining partial sequence information such as the identity of one or more nucleotides within a polynucleotide, determining the identity and order of nucleotides within a polynucleotide, etc.

Nucleic acid sequence information can be an important data set for medical and academic research endeavors. Sequence information can facilitate medical studies of active disease and genetic disease predispositions, and can assist in rational design of drugs (e.g., targeting specific diseases, avoiding unwanted side effects, improving potency, and the like). Sequence information can also be a basis for genomic and evolutionary studies and many genetic engineering applications. Reliable sequence information can be critical for other uses of sequence data, such as paternity tests, criminal investigations and forensic studies.

Sequencing technologies and systems, such as, for example, those provided by Applied Biosystems/Life Technologies (SOLiD Sequencing System), Illumina, and 454 Life Sciences can provide high throughput DNA/RNA sequencing capabilities to the masses. Applications which may benefit from these sequencing technologies include, but are certainly not limited to, targeted resequencing, miRNA analysis, DNA methylation analysis, whole-transcriptome analysis, and cancer genomics research.

Sequencing platforms can vary from one another in their mode of operation (e.g., sequencing by synthesis, sequencing by ligation, pyrosequencing, etc.) and the type/form of raw sequencing data that they generate. However, attributes that are typically common to all these platforms is that the sequencing runs performed on the platforms tend to be expensive, take a considerable amount of time to complete, and generate large quantities of data.

### SUMMARY

In various embodiments, a processor can dynamically model and correct sequencing signal data to account for through-cycle build-up. The processor can use the corrected sequencing signal data to determine a call for the sequence data. These and other features are provided herein.

In various embodiments, a method can include performing first and second rounds of a sequencing reaction on a plurality of targets, and obtaining a first set and a second set of spectral data corresponding to the first round and the second round respectively. The method can further include determining a scaling factor based on the first and second sets of spectral data, applying the scaling factor to the second set of spectral data to obtain modified spectral data for the targets, and determining a call for the targets based on the modified spectral data.

A system can include a memory circuit and a processor in communication with the memory circuit. The memory circuit can be configured to store a first and second set of spectral data. The first set of spectral data corresponding to a first round of a sequencing reaction performed on a plurality of targets, and the second set of spectral data corresponding to a second round of a sequencing reaction performed on the targets. The processor can be configured to determine a scaling factor based on the first and second sets of spectral data, apply the scaling factor to the second set of spectral data to obtain modified spectral data for the targets, and determine a call for the targets based on the modified spectral data.

A computer program product can include a non-transitory computer-readable storage medium whose contents include a program with instructions to be executed on a processor. The instructions can include instructions for obtaining a first set of spectral data, the first set of spectral data corresponding to a first round of a sequencing reaction performed on a plurality of targets, and instructions for obtaining a second set of spectral data, the second set of spectral data corresponding to a second round of a sequencing reaction performed on the targets. The instructions can further include instructions for determining a scaling factor based on the first and second sets of spectral data, instructions for applying the scaling factor to the second set of spectral data to obtain a modified spectral data for the targets, and instructions for determining a call for the targets based on the modified spectral data.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts an exemplary graph displaying the error rate as a function of sequencing cycle.

FIG. 2 is a flow diagram illustrating an exemplary embodiment of a method of modeling and correcting sequencing signal data.

FIG. 3 depicts an exemplary graph displaying the error rate as a function of sequencing cycle.

FIGS. 4A and 4B depict exemplary graphs displaying observed and corrected signals.

FIG. 5 is a block diagram illustrating an exemplary sequencing system.

FIG. 6 is a block diagram illustrating an exemplary computer system.

FIGS. 7A and 7B depict exemplary graphs displaying improvements to the error rate and mapping after correction.

FIG. 8 depicts an exemplary graph displaying mapping accuracy before and after use of residual correction for reverse reads.

FIG. 9 depicts an exemplary graph displaying the error rate as a function of position before and after use of residual correction for reverse reads.

FIG. 10 depicts an exemplary graph displaying mapping accuracy before and after use of residual correction for reverse reads.

FIG. 11 depicts an exemplary graph displaying the error rate as a function of position before and after use of residual correction for reverse reads.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts.

### DESCRIPTION OF VARIOUS EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As utilized in accordance with the embodiments provided herein, the following terms, unless otherwise indicated, shall be understood to have the following meanings:

As used herein, “a” or “an” means “at least one” or “one or more”.

The phrase “next generation sequencing” refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, pyrosequencing, and sequencing by hybridization. More specifically, the SOLiD Sequencing System of Life Technologies Corp. provides massively parallel sequencing with enhanced accuracy. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. Patent Publication 2011/0124111, entitled “Low-Volume Sequencing System and Method of Use,” filed on Aug. 31, 2010, and U.S. Patent Publication 2011/0128545, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

The phrase “ligation cycle” refers to a step in a sequence-by-ligation process where a probe sequence is ligated to a primer or another probe sequence.

The phrase “color call” refers to an observed dye color that results from the detection of a probe sequence after a ligation cycle of a sequencing run. Similarly, other “calls” refer to the distinguishable feature observed.

The phrase “synthetic bead” or “synthetic control” refers to a bead or some other type of solid support having multiple copies of synthetic template nucleic acid molecules attached to the bead or solid support. A linker sequence can be used to attach the synthetic template to the bead.

The phrase “fragment library” refers to a collection of nucleic acid fragments, wherein one or more fragments are used as a sequencing template. A fragment library can be generated, for example, by cutting or shearing, either enzymatically, chemically or mechanically, a larger nucleic acid into smaller fragments. Fragment libraries can be generated from naturally occurring nucleic acids, such as bacterial nucleic acids. Libraries comprising similarly sized synthetic nucleic acid sequences can also be generated to create a synthetic fragment library.

The phrase “mate-pair library” refers to a collection of nucleic acid sequences comprising two fragments having a relationship, such as by being separated by a known number of nucleotides. Mate pair fragments can be generated by cutting or shearing, or they can be generated by circularizing fragments of nucleic acids with an internal adapter construct and then removing the middle portion of the nucleic acid fragment to create a linear strand of nucleic acid comprising the internal adapter with the sequences from the ends of the nucleic acid fragment attached to either end of the internal adapter. Like fragment libraries, mate-pair libraries can be generated from naturally occurring nucleic acid sequences. Synthetic mate-pair libraries can also be generated by attaching synthetic nucleic acid sequences to either end of an internal adapter sequence.

The phrase “synthetic nucleic acid sequence” and variations thereof refers to a synthesized sequence of nucleic acid. For example, a synthetic nucleic acid sequence can be generated or designed to follow rules or guidelines. A set of synthetic nucleic acid sequences can, for example, be generated or designed such that each synthetic nucleic acid sequence comprises a different sequence and/or the set of synthetic nucleic acid sequences comprises every possible variation of a set-length sequence. For example, a set of 64 synthetic nucleic acid sequences can comprise each possible combination of a 3 base sequence, or a set of 1024 synthetic nucleic acid sequences can comprise each possible combination of a 5 base sequence.

The phrase “control set” refers to a collection of nucleic acids each having a known sequence and physical properties wherein there is a plurality of differing nucleic acid sequences. A control set can comprise, for example, nucleic acids associated with a solid support. In some embodiments a control set can comprise a set of solid supports having a number of nucleic acid sequences attached thereto. Control sets can also comprise a solid support having a collection of nucleic acids attached thereto, such that each of the differing nucleic acid sequences is located at a substantially distinct location on the solid support, and sets of solid supports each having a substantially uniform set of nucleic acids associated therewith. The source of the nucleic acid sequences can be synthetically derived nucleic acid sequences or naturally occurring nucleic acid sequences. The nucleic acid sequences, either naturally occurring or synthetic, can be provided, for example, as a fragment library or a mate-pair library, or as the analogous synthetic libraries. The nucleic acid sequences can also be in other forms, such as a template comprising multiple inserts and multiple internal adapters. Other forms of nucleic acid sequences can include concatenates.

The term “subset” refers to a grouping of synthetic nucleic acid sequences by a common characteristic. For example, a subset can comprise all of the synthetic nucleic acid sequences in a control set that exhibit the same color call in a first ligation cycle.

The term “template” and variations thereof refer to a nucleic acid sequence that is a target of nucleic acid sequencing. A template sequence can be attached to a solid support, such as a bead, a microparticle, a flow cell, or other surface or object. A template sequence can comprise a synthetic nucleic acid sequence. A template sequence also can include an unknown nucleic acid sequence from a sample of interest and/or a known nucleic acid sequence.

The phrase “template density” refers to the number of template sequences attached to each individual solid support.

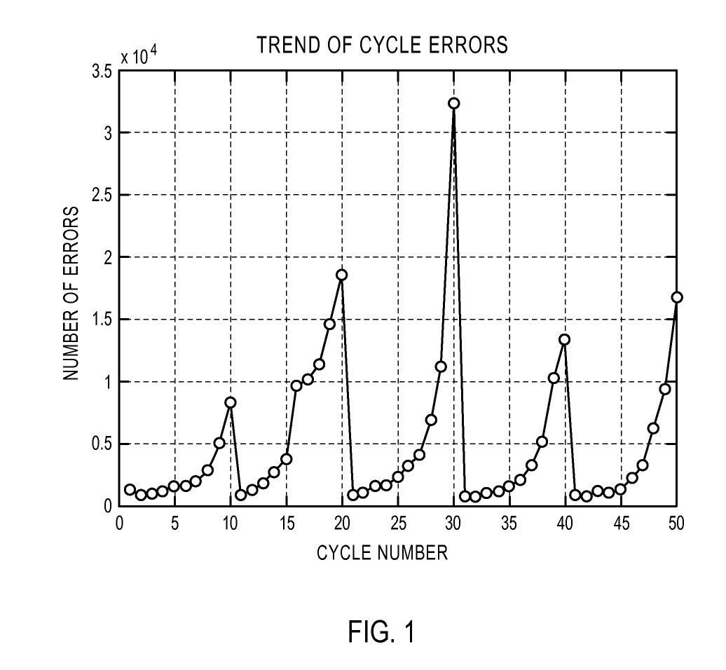

Next generation sequencing platforms are rapidly evolving to enable ultra-high throughput DNA sequencing while reducing the sequencing cost. However, it has been observed that later round sequencing cycles can have much higher error rate than earlier sequencing cycles. For example, see FIG. 1.

One of the factors contributing to such phenomena is through-cycle residual build-up, which results in the change of bead intensities captured by the instrument camera. Through cycle residual build-up can be attributed to inefficiencies in the chemical reactions involved in the sequencing process. During each cycle, a portion of the target molecules may not react completely, resulting in a subpopulation of target molecules that is behind the main population of target molecules.

For example, a labeled nucleotide or a labeled oligonucleotide probe may not be incorporated at a particular target molecule during a sequencing cycle. For example, the nucleotide or the oligonucleotide probe may not bind to a particular target molecule, ligation of the oligonucleotide probe may not occur, or a nucleotide may not be incorporated. While the labeled nucleotide or the labeled oligonucleotide probe may be incorporated in a subsequent sequencing cycle, the signal associated with the particular target molecule may not be reporting on the same sequence position as the main population of target molecules.

In another example, a label or a blocking moiety may not be removed during a current sequencing cycle, thus preventing the incorporation of the next labeled nucleotide or oligonucleotide probe in a subsequent sequencing cycle. If the label remains into the next sequencing cycle, the signal associated with the particular target molecule can report again on the sequence of the current position, rather than the subsequent position that the signal from the main population of target molecules will be reporting. Further, while the chemistry may be completed in the subsequent sequencing cycle, the signal associated with the particular target molecule can continue to lag the main population of target molecules.

Various embodiments of an efficient residual correction algorithm for color call improvement are provided herein. The algorithm can model the bead intensity at a given cycle as a function of the underlying bead intensity and residual effect from previous cycle. In some embodiments, the method can increase perfect matching and system accuracy by reducing errors for later ligation cycles. In some embodiments, the system also increases total matching throughput, while more significant improvement can be predicted for longer reads runs.

In various embodiments, a computer implemented method can dynamically model and correct sequencing signal data to account for the residual effect to improve a color call or a base call. The sequencing signal data can include multi-channel intensity data, such as intensity data for two or more fluorescent reporters. The corrected sequencing signal data can be used to determine color calls or base calls for the sequencing data.

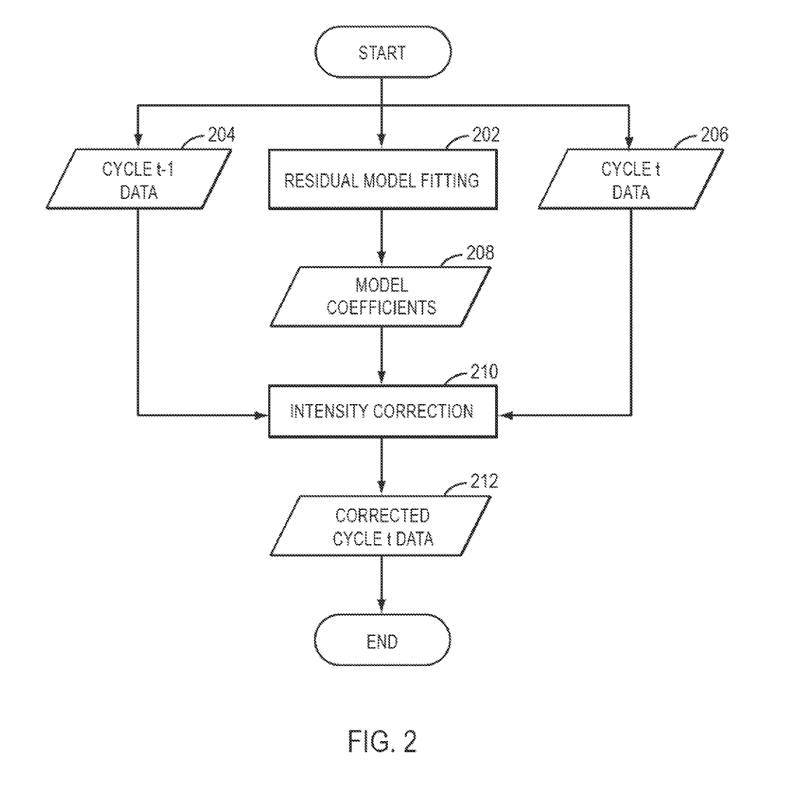

FIG. 2 illustrates a flow diagram of a method for correcting the multi-channel intensity data. At 202, residual model fitting utilizes cycle t−1 data 204 and cycle t data 206 to determine model coefficients 208. At 210, the model cycle t−1 data 204, cycle t data 206, and the model coefficients 208 are used to correct the correct the intensity for the samples, resulting in corrected cycle t data 212. The corrected cycle t data 212 can be used to improve color or base calling for the sequencing cycle.

In various embodiments, the corrected intensities can improve the sequencing results by increasing color calling or base calling accuracy. In various embodiments, the algorithm can be result in up to about 10% and about 50% throughput increase for total match and perfect match respectively. Further, increased color calling or base calling accuracy can increase the number of samples that can be called in a given cycle and can increase the number of cycles that can provide usable data for a given sample.

In various embodiments, the modeling and correction can be performed concurrent with sequencing. For example, the modeling and correction can be performed on the data for a sequencing cycle once the data is obtained but prior to the data for a subsequent cycle being available, such as while sequencing chemistry or data collection of the subsequent cycle is being performed. In other particular embodiments, the modeling and correction can be performed batch-wise, such as when sequencing signal data is available for multiple sequencing cycles. For example, the modeling and correction of the sequencing signal data can be performed for data from multiple cycles after the completion of a sequencing run, after the completion of a sequencing round, or after completion of multiple cycles of a sequencing round.

In various embodiments, sequencing signal data from a first sequencing cycle of a sequencing round, such as incorporation of a first nucleotide during a round of sequencing-by-synthesis, or ligation of a first probe during a round of sequencing-by-ligation, may not include an observable through-cycle build-up component due to the absence of prior rounds. As such, modeling and correction of the sequencing signal data may not occur for the first sequencing cycle.

$$b {λ}^{k} [ \begin{matrix}{S}_{t , 1}^{k} \\ {S}_{t , 2}^{k} \\ {S}_{t , 3}^{k} \\ {S}_{t , 4}^{k}\end{matrix} ] + d [ \begin{matrix}{α}_{1} {I}_{t - 1 , 1}^{k} \\ {α}_{2} {I}_{t - 1 , 2}^{k} \\ {α}_{3} {I}_{t - 1 , 3}^{k} \\ {α}_{4} {I}_{t - 1 , 4}^{k}\end{matrix} ] + [ \begin{matrix}{c}_{1} \\ {c}_{2} \\ {c}_{3} \\ {c}_{4}\end{matrix} ] = [ \begin{matrix}{I}_{t , 1}^{k} \\ {I}_{t , 2}^{k} \\ {I}_{t , 3}^{k} \\ {I}_{t , 4}^{k}\end{matrix} ] \qquad Equation 1$$

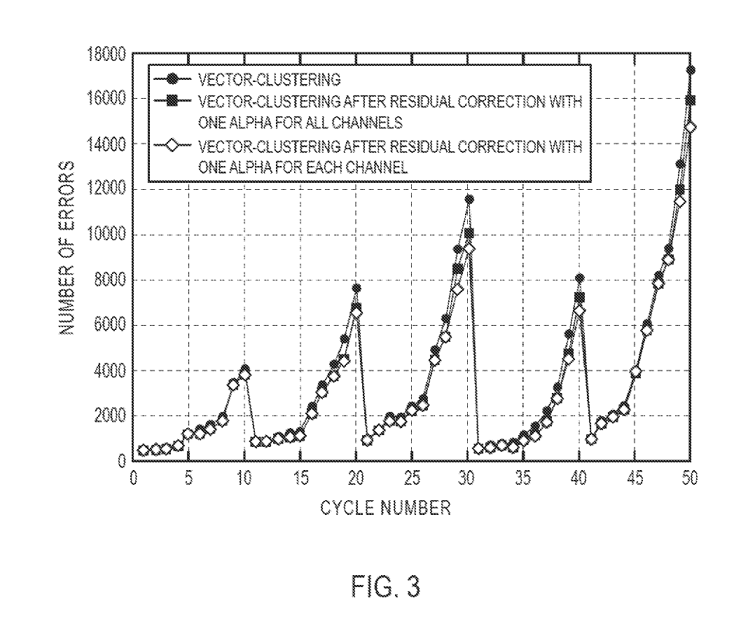

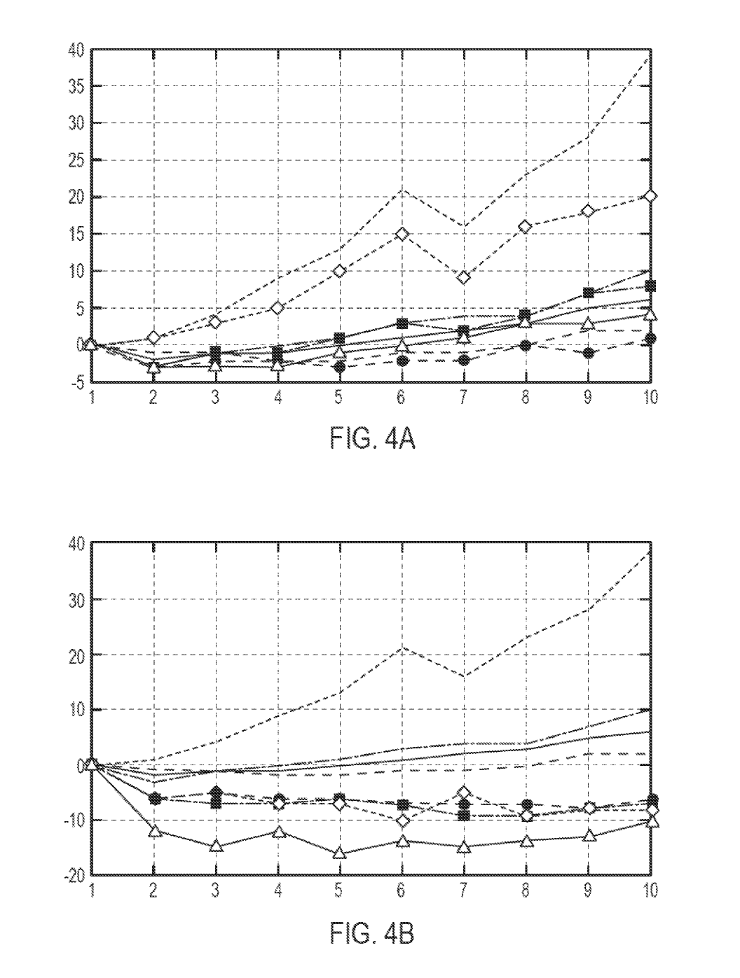

In various embodiments, as shown in Equation 1, the multi-channel intensity data at a given cycle can be modeled as the sum of three components: 1) the underlying theoretical intensity vector at the current cycle, 2) the residual effect from the immediate previous cycle as the product of the residual coefficients and the intensity vector of the previous cycle, and 3) a vector term representing the background difference between the two cycles. Specifically, in Equation 1, d is a decay coefficient, λ^{k} is a template concentration for bead k, αᵢ is a residual coefficient for channel i, cᵢ is a background level difference for channel i, S^{k}_{t,i} is an initial color call result for bead k, channel i at cycle t, I^{k}_{t,i} is an intensity value for bead k, channel i at cycle t, and b is a scale factor. In particular embodiments, it may not be necessary to solve for d and αᵢ separately. In particular embodiments, the coefficient λ^{k} (target-dependant) can be replaced by λⱼ (channel-dependant, j=1,2,3,4) or λ (independent of bead or channel). Among the three terms, both the residual-coefficients and the background difference terms can be channel-independent or channel-dependent, an example of which being demonstrated by FIG. 3 and FIG. 4. FIG. 3 shows the number of errors per cycle without residual correct, with residual correct with a channel independent residual coefficient α, and with residual correction with a channel depended residual coefficient αᵢ. FIG. 4 shows the number of errors per cycle before (solid lines) and after (lines with circles) residual correction. The model used for residual correction in the top panel does not utilize a background difference term, whereas the model used for the residual correction in the bottom panel utilizes a background difference term. The model can be solved mathematically through least square fitting technique, and the residual and the background difference can be subtracted from the current cycle to recover the underlying intensity. The corrected intensity can be used to determine more accurate color calls. In practice, the workflow can include three steps: 1) a chosen color caller can feed the initial color call values into the model; 2) the underlying intensity values can be recovered by the model; and 3) the recovered intensity values can be fed into the color caller to refine the color calls. In particular embodiments, the workflow can be iteratively repeated until the refined color calls converge.

In some embodiments, to improve the computation efficiency, only a subset of the samples in each panel may be used for model fitting and the solved model parameters can be applied to all the beads in the same panel. In some embodiments, the subset of samples can be randomly selected. In other embodiments in which the set of samples can include both unknown target sequences and known control sequences, the subset of samples can be selected from the known control sequences. In some embodiments, to improve the modeling accuracy, beads can be excluded from being sampled during modeling if they have repeating color call sequences (previous and current), since such sequences have more chance of being residual induced errors.

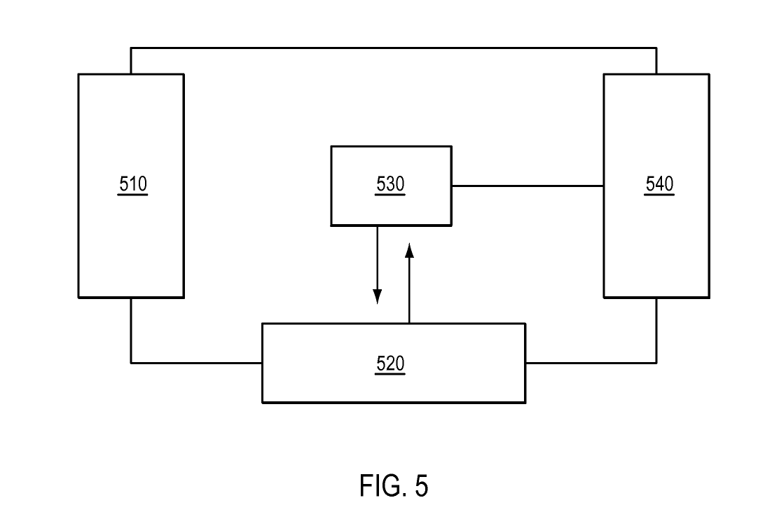

Various embodiments of platforms for next generation sequencing can include components as displayed in the block diagram of FIG. 5. According to various embodiments, sequencing instrument can include a fluidic delivery and control unit 510, a sample processing unit 520, a signal detection unit 530, and a data acquisition, analysis and control unit 540. Various embodiments of instrumentation, reagents, libraries and methods used for next generation sequencing are described in U.S. Patent Application Publication No. 2007/066931 and U.S. Patent Application Publication No. 2008/003571, which applications are incorporated herein by reference. Various embodiments of instrument can provide for automated sequencing that can be used to gather sequence information from a plurality of sequences substantially simultaneously, such as in parallel.

In various embodiments, the sample processing unit 520 can include a sample chamber, such as flow cell, a substrate, a micro-array, a multi-well tray, or the like. The sample processing unit 520 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 530 can include an imaging or detection sensor. For example, the imaging or detection sensor can include a CCD, a CMOS, an ion sensor, such as an ion sensitive layer overlying a CMOS, a current detector, or the like. The signal detection unit 530 can include an excitation system to cause a probe, such as a fluorescent dye, to emit a signal. The expectation system can include an illumination source, such as arc lamp, a laser, a light emitting diode (LED), or the like. In particular embodiments, the signal detection unit 530 can include optics for the transmission of light from an illumination source to the sample or from the sample to the imaging or detection sensor. Alternatively, the signal detection unit 530 may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current can be detected as a nucleic acid passes through a nanopore without the need for an illumination source.

In various embodiments, data acquisition analysis and control unit 540 can monitor various system parameters. The system parameters can include temperature of various portions of instrument 500, such as sample processing unit or reagent reservoirs, volumes of various reagent, the status of various system subcomponents, such as a manipulator, a stepper motor, a pump, or the like, or any combination thereof.

It will be appreciated by one skilled in the art that various embodiments of instrument 500 can be used to practice variety of sequencing methods including ligation-based methods, sequencing by synthesis, single molecule methods, nanopore sequencing, and other sequencing techniques. Ligation sequencing can include single ligation techniques, or change ligation techniques where multiple ligations are performed in sequence on a single primary. Sequencing by synthesis can include the incorporation of dye labeled nucleotides, chain termination, ion/proton sequencing, pyrophosphate sequencing, or the like. Single molecule techniques can include continuous sequencing, where the identity of the nuclear type is determined during incorporation without the need to pause or delay the sequencing reaction, or staggered sequence, where the sequencing reactions is paused to determine the identity of the incorporated nucleotide.

In various embodiments, the sequencing instrument 500 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 500 can obtain the sequence information from a single nucleic acid molecule or from a group of substantially identical nucleic acid molecules.

The sequencing instrument 500 can operate on a sample, a control, or a combination thereof. The sample can include a nucleic acid with an unknown sequence. The control can include a nucleic acid with a known sequence, and can include or be derived from a synthetic or natural nucleic acid. The sample or control nucleic acid can be attached to a solid or semi-solid support. Examples of a support can include a bead, a slide, a surface of a flow cell, a matrix on a surface, a surface of a well, or the like. In particular embodiments, the surface may include multiple nucleic acids with a substantially identical sequence grouped together. For example, a bead can have a population of substantially identical nucleic acids. The sequencing instrument may determine sequence information from multiple beads simultaneously in a parallel fashion. In another example, a surface can be populated with multiple clusters of nucleic acids, with each cluster including a population of substantially identical nucleic acids.

In the various examples and embodiments described herein, a system for sequencing nucleic acid samples can include a sequencing instrument and a processor in communication with the sequencing instrument. In some embodiments, sequencing instruments can be in communication with other sequencing instruments as well as with processors, and processors can be in communication with other processors as well as with sequencing instruments. Communication between and among sequencing instruments and processors can take many forms known the skilled artisan, including direct or indirect and physical, electronic/electromagnetic), or otherwise functional (e.g., information can be transferred via wires, fiber optics, wireless systems, networks, internet, hard drives or other memory devices, and the like).

In various embodiments, a sequencing instrument can perform sequencing by successive rounds of extension, ligation, detection, and cleavage, as described in more detail in PCT Publication No. WO 2006/084132, entitled “Reagents, Methods, and Libraries for Bead-Based Sequencing,” international filing date Feb. 1, 2006, the entirety of which being incorporated herein by reference thereto. The successive rounds can proceed from a 5′-end of a target sequence or from the 3′-end of the target sequence. Additionally, the successive rounds can proceed from a free end of the template towards a support, or from the support towards a free end of the template.

By way of an example, a template containing binding region and polynucleotide region of unknown sequence can be attached to a support, e.g., a bead. An initializing oligonucleotide with an extendable terminus can be annealed to binding region. The extendable terminus can include a free 3′-OH group when extending from a 5′→3′ direction or a free 5′ phosphate group when extending from a 3′→5′ direction. Extension probe can be hybridized to the template in polynucleotide region. Nucleotides of the extension probe can form a complementary base pair with unknown nucleotides in the template. Extension probe can be ligated to the initializing oligonucleotide, such as, for example, using T4 ligase. Following ligation, the label attached to extension probe can be detected. The label can correspond to the identity of one or more nucleotides of the template. Thus the nucleotides can be identified as the nucleotide complementary to the nucleotides of the template. In various embodiments, identification of the nucleotides in subsequence ligation cycles can be improved through the use of algorithms to dynamically model and correct the residual effect, as described herein. Extension probe can then cleaved at a phosphorothiolate linkage such as, for example, using AgNO₃ or another salt that provides Ag⁺ ions, resulting in an extended duplex. Cleavage can leave a phosphate group at the 3′ end of the extended duplex for extension in the 5′→3′ direction, or an extendable monophosphate group at the 5′ end of the extend duplex for extension in the 3′→5′ direction. For extension in the 5′→3′ direction, phosphatase treatment can be used to generate an extendable probe terminus on the extended duplex. The process can be repeated for a desired number of cycles.

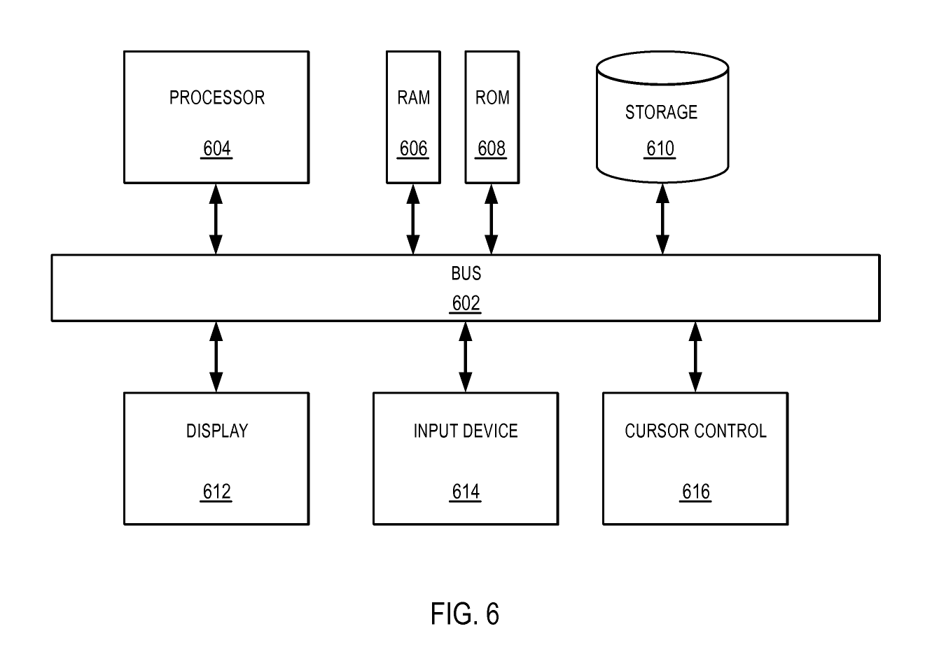

FIG. 6 is a block diagram that illustrates a computer system 600, upon which embodiments of the present teachings can be implemented. Examples of a computer system 600 can include a server system or client system, such as desktop or laptop, or a mobile or handheld system, such as a PDA, smartphone, tablet, or the like. Computer system 600 can be a general purpose computer, such as a general-purpose computer program performs specific functions, or a special-purpose computer.

Computer system 600 can include a bus 602 or other communication mechanism for communicating information, and a processor 604 coupled with bus 602 for processing information. In various embodiments, the processor 604 can include a Central Processing Unit (CPU), such as a coreDuo, a Nehalem, an Athlon, an Opteron, a PowerPC, or the like, a Graphics processing unit (GPU), such as the GeForce, Tesla, Radeon HD, or the like, an Application-specific integrated circuit (ASIC), a Field programmable gate array (FPGA), or the like. In various embodiments, the processor 604 can include a single core processor or a multi-core processor. Additionally, multiple processors can be coupled together to perform tasks in parallel.

Computer system 600 can also include a memory 606, which can be a random access memory (RAM) or other dynamic storage device, coupled to bus 602. Memory 606 can store data, such as sequence information, and instructions to be executed by processor 604. Memory 606 can also be used for storing temporary variables or other intermediate information during execution of instructions to be executed by processor 604. Computer system 600 can further include a read-only memory (ROM) 608 or other static storage device coupled to bus 602 for storing static information and instructions for processor 604. A storage device 610, such as a magnetic disk, an optical disk, a flash memory, or the like, can be provided and coupled to bus 602 for storing information and instructions.

Computer system 600 can be coupled by bus 602 to display 612, such as a cathode ray tube (CRT) or liquid crystal display (LCD), for displaying information to a computer user. An input device 614, such as a keyboard including alphanumeric and other keys, can be coupled to bus 602 for communicating information and commands to processor 604. Cursor control 616, such as a mouse, a trackball, a trackpad, or the like, can communicate direction information and command selections to processor 604, such as for controlling cursor movement on display 612. The input device can have at least two degrees of freedom in at least two axes that allows the device to specify positions in a plane. Other embodiments can include at least three degrees of freedom in at least three axes to allow the device to specify positions in a space. In additional embodiments, functions of input device 614 and cursor 616 can be provided by a single input devices such as a touch sensitive surface or touch screen.

Computer system 600 can perform the present teachings. Consistent with certain implementations of the present teachings, results are provided by computer system 600 in response processor 604 executing one or more sequences of one or more instructions contained in memory 606. Such instructions may be read into memory 606 from another computer-readable medium, such as storage device 610. Execution of the sequences of instructions contained in memory 606 can cause processor 604 to perform the processes described herein. Alternatively, hard-wired circuitry may be used in place of or in combination with software instructions to implement the present teachings. Thus, implementations of the present teachings are not limited to any specific combination of hardware circuitry and software.

The term “computer-readable medium” as used herein refers to any media that participates in providing instructions to processor 604 for execution. Such a medium may take many forms, including but not limited to, nonvolatile memory, volatile memory, and transmission media. Nonvolatile memory includes, for example, optical or magnetic disks, such as storage device 610. Volatile memory includes dynamic memory, such as memory 606. Transmission media includes coaxial cables, copper wire, and fiber optics, including the wires that comprise bus 602. Non-transitory computer readable medium can include nonvolatile media and volatile media.

Common forms of non-transitory computer readable media include, for example, floppy disk, flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, and other memory chips or cartridge or any other tangible medium from which the computer can read.

Various forms of computer readable media may be involved in carrying one or more sequences of one or more instructions to processor 604 for execution. For example the instructions may initially be stored on the magnetic disk of a remote computer. The remote computer can load the instructions into its dynamic memory and send instructions over a network to computer system 600. A network interface coupled to bus 602 can receive the instructions and place the instructions on bus 602. Bus 602 can carry the instructions to memory 606, from which processor 604 can retrieve and execute the instructions. Instructions received by memory 606 may optionally be stored on storage device 610 either before or after execution by processor 604.

In accordance with various embodiments, instructions configured to be executed by processor to perform a method are stored on a computer readable medium. The computer readable medium can be a device that stores digital information. For example, a computer readable medium can include a compact disc read-only memory as is known in the art for storing software. The computer readable medium is accessed via processor suitable for executing instructions configured to be executed.

In a first aspect, a method can include performing a first round of a sequencing reaction on a plurality of targets, and obtaining a first set of multi-channel intensity data for the targets. Each target can include a substantially homogenous population of nucleic acids. The method can further include performing a second round of a sequencing reaction on the targets, and obtaining a second set of multi-channel intensity data for the targets. The method can further include determining a correction factor based on the first and second sets of multi-channel intensity data, applying the correction factor to the second set of multi-channel intensity data to obtain a corrected multi-channel intensity for each target, and determining a color call or a base call for the targets based on the corrected multi-channel intensity.

In a second aspect, a system can include a memory and a processor. The memory can be configured to store a first and a second set of multi-channel intensity data. The first set of multi-channel intensity data can correspond to a first round of a sequencing reaction performed on a plurality of targets. The second set of multi-channel intensity data can correspond to a second round of a sequencing reaction performed on the targets. Each target can include a substantially homogenous population of nucleic acids. The processor can be configured to determine a correction factor based on the first and second sets of multi-channel intensity data, apply the correction factor to the second set of multi-channel intensity data to obtain a corrected multi-channel intensity for each target, and determine a color call or a base call for the targets based on the corrected multi-channel intensity.

In a third aspect, a computer program product can include a non-transitory computer-readable storage medium whose contents include a program with instructions being executed on a processor. The instructions can include instructions for obtaining a first set of multi-channel intensity data. The first set of multi-channel intensity data can correspond to a first round of a sequencing reaction performed on a plurality of targets. Each target can include a substantially homogenous population of nucleic acids. The instructions can further include instructions for obtaining a second set of multi-channel intensity data. The second set of multi-channel intensity data can correspond to a second round of a sequencing reaction performed on the targets. The instructions can further include instructions for determining a correction factor based on the first and second sets of multi-channel intensity data, instructions for applying the correction factor to the second set of multi-channel intensity data to obtain a corrected multi-channel intensity for each target, and instructions for determining a color call or a base call for the targets based on the corrected multi-channel intensity.

In various embodiments, the corrected multi-channel intensity can be a function of the second set of multi-channel intensity data, a background difference between the first and second set of multi-channel intensity data, and a product of the correction factor and the first set of multi-channel intensity data.

In various embodiments, determining the correction factor can rely upon the multi-channel intensity data for a subset of the targets.

In particular embodiments, the plurality of the targets can include a set of samples and a set of controls. Each target within the set of samples can include a substantially homogenous population of unknown nucleic acids and each target within the set of controls can include a substantially homogenous population of control nucleic acids. The subset of the targets used for determining the correction factor can correspond to the set of controls.

In particular embodiments, determining the correction factor can include determining an initial color call or base call based on the second set of multi-channel intensity data for the subset of the targets, and modeling a correction factor based on the initial color call and the first and second sets of multi-channel intensity data.

In particular embodiments, determining the corrector factor further includes iteratively performing the steps of determining the correction factor for the subset of targets, applying the correction factor to the second set of multi-channel intensity data to obtain corrected intensity data for the subset of targets, determining a color call or base call for the subset of targets, and using the color call or base call to further refine the correction factor until the color call or base call for the subset of targets converges.

In various embodiments, the targets include beads with bound nucleic acids molecules, colonies of nucleic acids molecules bound to a support, clusters of nucleic acids molecules bound to a support, DNA nanoballs bound to a support, or a combination thereof.

### EXAMPLES

FIG. 3 illustrates exemplary data showing a comparison of the number of errors per cycle when no residual correction is performed, when residual correction is performed using a channel independent α, and when residual correction is performed using a channel independent α. The use of residual correction with a channel independent α resulting in a 7.6% reduction in the number of errors per cycle compared to no residual correction. Residual correction with a channel dependent α resulted in 11.4% reduction of errors compared to no residual correction.

FIG. 4 illustrates exemplary data showing a comparison of the number of errors per cycle when no residual correction is performed (solid lines), when residual correction is performed without a background difference term (lines with circles in the top panel), and when residual correction is performed with a background difference term (lines with circles in the bottom panel). The use of the background difference term provides significant improvement over residual correction without account for the background difference.

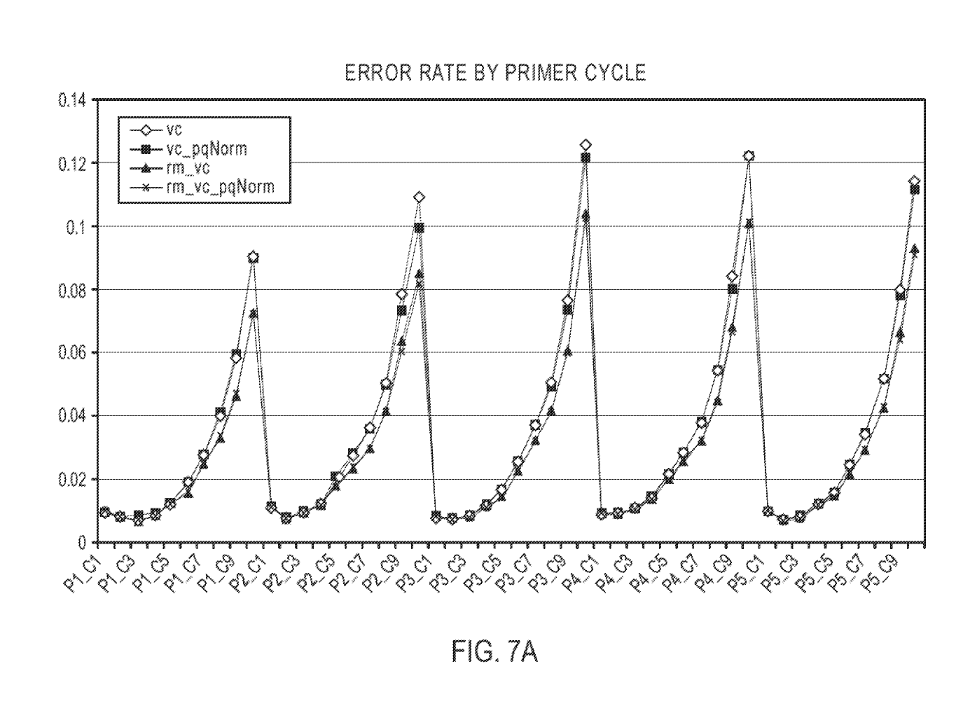

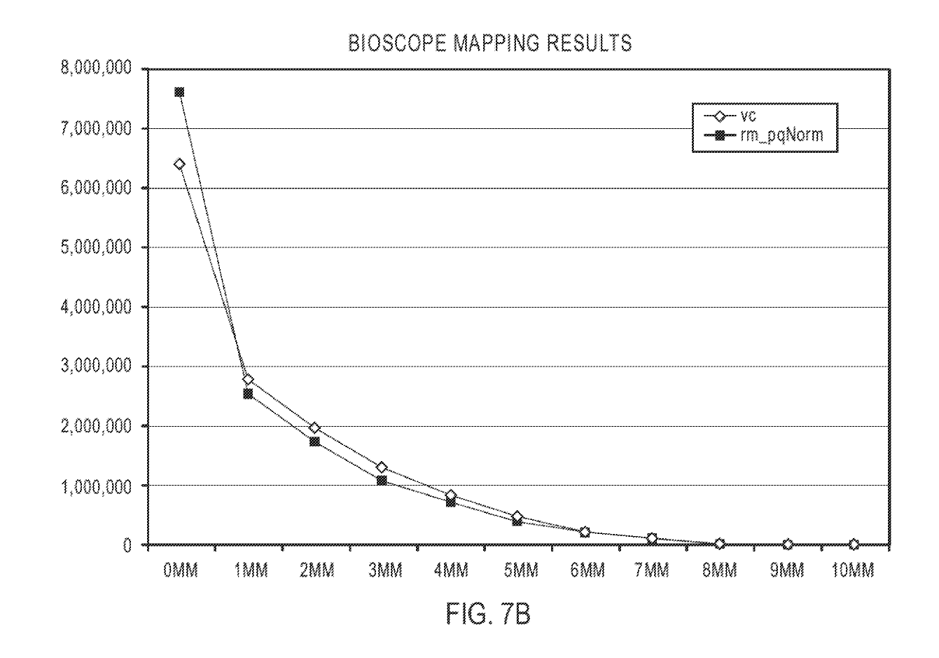

FIG. 7 illustrates exemplary data showing the improvement in the errors per cycle and the mapping results provided when using residual correction.

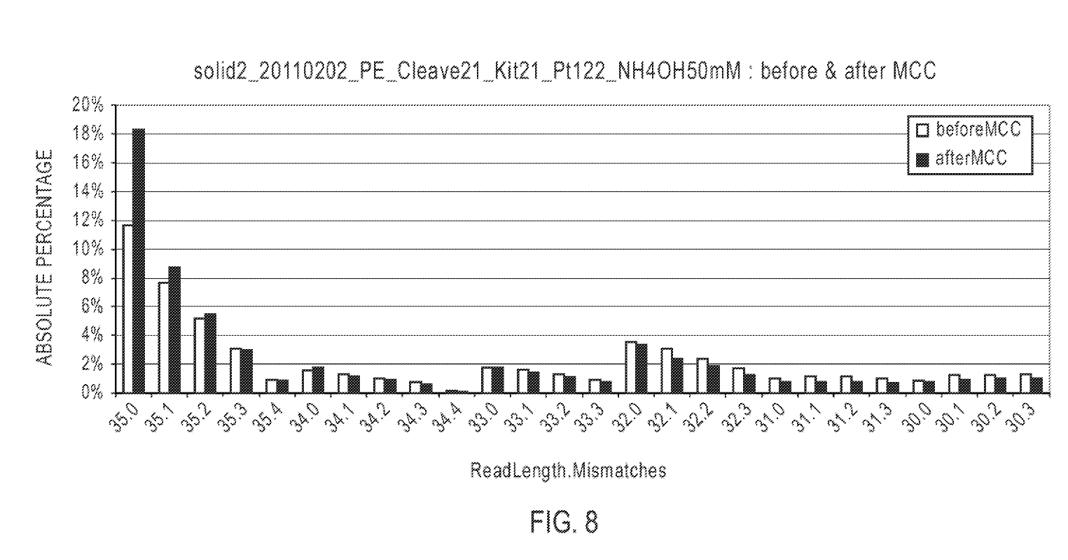

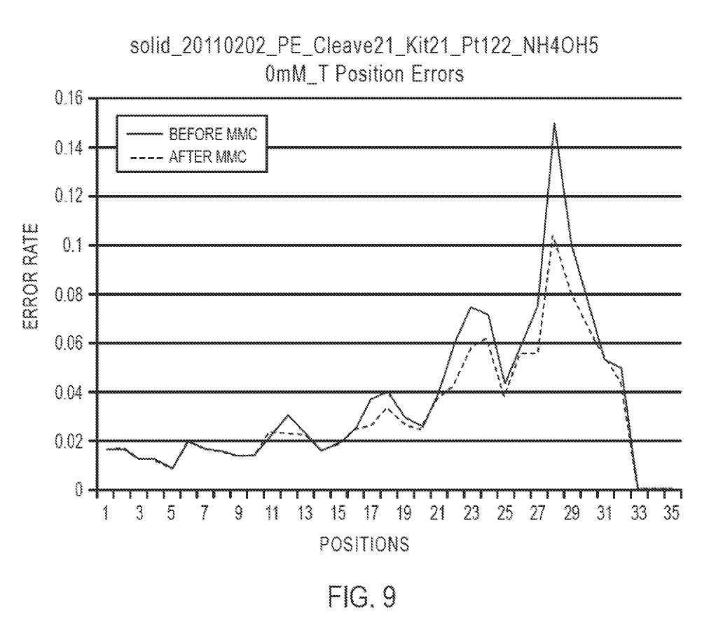

FIG. 8 illustrates exemplary reverse read data showing a comparison of the mapping accuracy before and after the use of residual correction. Total matching improves from 58.98% without residual correction to 61.84% with residual correction. Similarly, accuracy improves from 96.19% without residual correction to 96.78% with residual correction. FIG. 9 shows for the same exemplary reverse read data that the error rate as a function of position in the nucleic acid sequence improves with the use of residual correction.

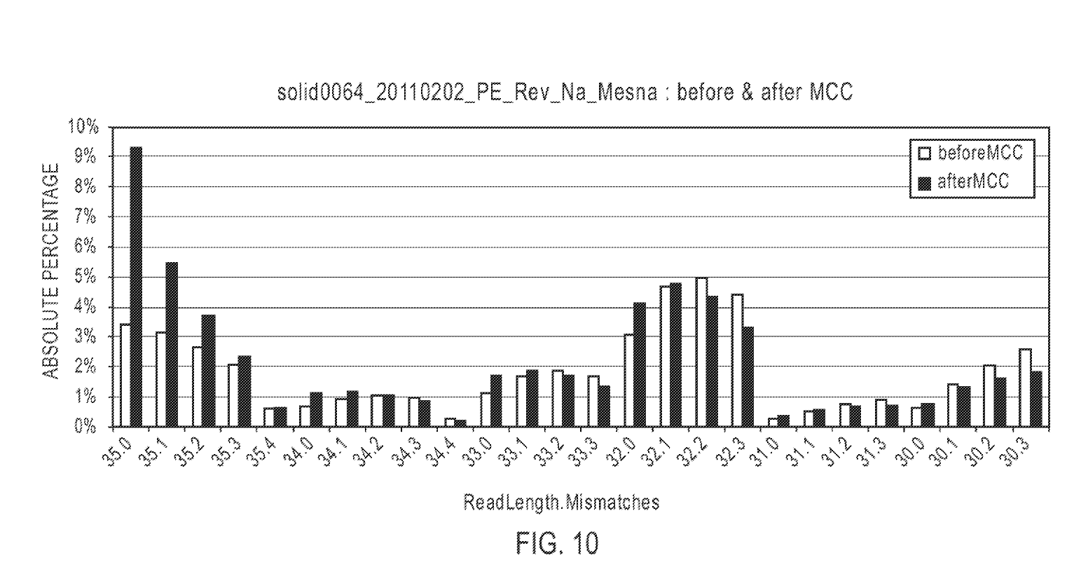

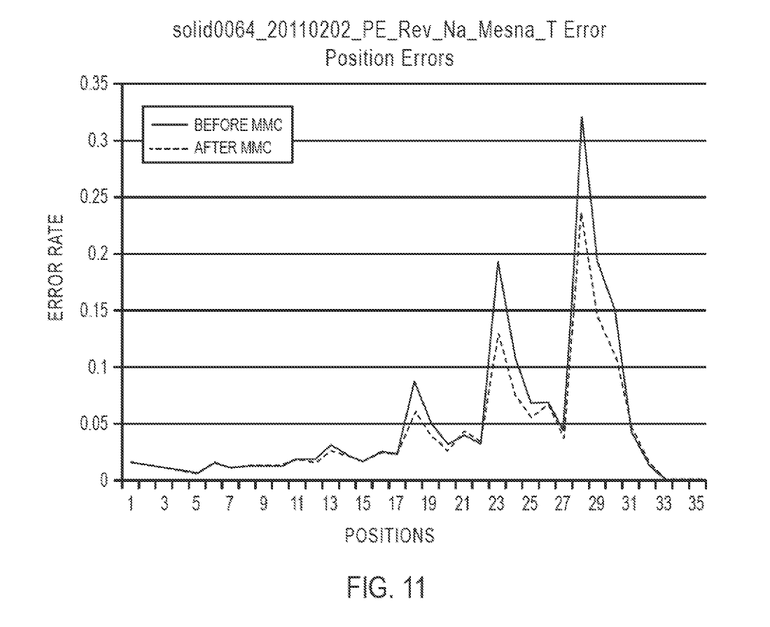

FIG. 10 illustrates additional exemplary reverse read data showing a comparison of the mapping accuracy before and after the use of residual correction. Total matching improves from 48.20% without residual correction to 56.59% with residual correction. Similarly, accuracy improves from 94.73% without residual correction to 95.80% with residual correction. FIG. 11 shows for the same exemplary reverse read data that the error rate as a function of position in the nucleic acid sequence improves with the use of residual correction.

While the principles of the present teachings have been described in connection with specific embodiments of control systems and sequencing platforms, it should be understood clearly that these descriptions are made only by way of example and are not intended to limit the scope of the present teachings or claims. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalents.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method comprising:
performing a first round of a sequencing reaction on a plurality of targets;
obtaining a first set of spectral data corresponding to the first round;
performing a second round of a sequencing reaction on the targets;
obtaining a second set of spectral data corresponding to the second round;
determining a scaling factor based on the first and second sets of spectral data;
applying the scaling factor to the second set of spectral data to obtain modified spectral data for the targets; and
determining a call for the targets based on the modified spectral data.

2. The method of claim 1, wherein a target includes a substantially homogenous population of nucleic acids.

3. The method of claim 1, wherein the first and second sets of spectral data include multi-channel intensity data.

4. The method of claim 1, wherein the call is a base call, a color call, or a combination thereof.

5. The method of claim 1, wherein the first and second rounds of a the sequencing reaction include a ligation of a probe, a polymerization of a nucleotide, or a combination thereof.

6. The method of claim 1, wherein the modified spectral data is a function of the second set of spectral data, a background difference between the first and second set of spectral data, and a product of the scaling factor and the first set of spectral data.

7. The method of claim 1, wherein determining the scaling factor relies upon the spectral data for a subset of the targets.

8. The method of claim 7, wherein the plurality of the targets includes a set of samples and a set of controls, the targets of the set of samples including substantially homogenous populations of unknown nucleic acids and the targets of the set of controls including substantially homogenous populations of control nucleic acids, and the subset of the targets used for determining the correction factor corresponds to the set of controls.

9. The method of claim 7, wherein determining a factor includes determining an initial call based on the second set of multi-channel intensity data for the subset of the targets, and modeling a correction factor based on the initial color call and the first and second sets of spectral data.

10. The method of claim 9, wherein determining a scaling factor further includes iteratively performing the steps of determining the scaling factor for the subset of targets, applying the scaling factor to the second set of spectral data to obtain modified spectral data for the subset of targets, determining the call for the subset of targets, and using the call to refine the scaling factor until the call for the subset of targets converges.

11. The method of claim 1, wherein the targets include beads, colonies, clusters, DNA nanoballs, or a combination thereof.

12. A system comprising:
a memory circuit configured to store a first and second set of spectral data, the first set of spectral data corresponding to a first round of a sequencing reaction performed on a plurality of targets, the second set of spectral data corresponding to a second round of a sequencing reaction performed on the targets; and
a processor in communication with the memory circuit, the processor configured to:
determine a scaling factor based on the first and second sets of spectral data;
apply the scaling factor to the second set of spectral data to obtain modified spectral data for the targets; and
determine a call for the targets based on the modified spectral data.

13. The system of claim 12, wherein the modified spectral data is a function of the second set of spectral data, a background difference between the first and second set of spectral data, and a product of the scaling factor and the first set of spectral data.

14. The system of claim 12, wherein determining a scaling factor relies upon the spectral data for a subset of the targets.

15. The system of claim 14, wherein the plurality of the targets includes a set of samples and a set of controls, the targets of the set of samples include substantially homogenous populations of unknown nucleic acids and targets of the set of controls include substantially homogenous populations of control nucleic acids, and the subset of the targets used for determining the scaling factor corresponds to the set of controls.

16. The system of claim 14, wherein determining a scaling factor includes determining an initial call based on the second set of spectral data for the subset of the targets, and modeling a scaling factor based on the initial call and the first and second sets of spectral data.

17. The system of claim 16, wherein determining a scaling factor further includes iteratively performing the steps of determining the scaling factor for the subset of targets, applying the scaling factor to the second set of spectral data to obtain modified spectral data for the subset of targets, determining the call for the subset of targets, and using the call to refine the scaling factor until the call for the subset of targets converges.

18. The system of claim 12, wherein the targets include beads, colonies, clusters, DNA nanoballs, or a combination thereof.

19. A computer program product, comprising a non-transitory computer-readable storage medium whose contents include a program with instructions to be executed on a processor, the instructions comprising:
instructions for obtaining a first set of spectral data, the first set of spectral data corresponding to a first round of a sequencing reaction performed on a plurality of targets;
instructions for obtaining a second set of spectral data, the second set of spectral data corresponding to a second round of a sequencing reaction performed on the targets;
instructions for determining a scaling factor based on the first and second sets of spectral data;
instructions for applying the scaling factor to the second set of spectral data to obtain a modified spectral data for the targets; and
instructions for determining a call for the targets based on the modified spectral data.

20. The computer program product of claim 19, wherein the modified spectral data is a function of the second set of spectral data, a background difference between the first and second set of spectral data, and a product of the scaling factor and the first set of spectral data.

21. The computer program product of claim 19, wherein determining a scaling factor relies upon the spectral data for a subset of the targets.

22. The computer program product of claim 21, wherein the plurality of the targets includes a set of samples and a set of controls, the targets of the set of samples include substantially homogenous populations of unknown nucleic acids and the targets of the set of controls include substantially homogenous populations of control nucleic acids, and the subset of the targets used for determining the scaling factor corresponds to the set of controls.

23. The computer program product of claim 21, wherein determining the scaling factor includes determining an initial call based on the second set of spectral data for the subset of the targets, and modeling the scaling factor based on the initial call and the first and second sets of spectral data.

24. The computer program product of claim 23, wherein determining the scaling factor further includes iteratively performing the steps of determining the scaling factor for the subset of targets, applying the scaling factor to the second set of spectral data to obtain modified spectral data for the subset of targets, determining the call for the subset of targets, and using the call to refine the scaling factor until the call for the subset of targets converges.

25. The computer program product of claim 19, wherein the targets include beads, colonies, clusters, DNA nanoballs, or a combination thereof.
